(19) United States
(12) Patent Application Publication
(10) Pub. No.: US 2012/0010718 A1
(43) Pub. Date: Jan. 12, 2012

(54) PARTIAL ANKLE JOINT REPLACEMENT IMPLANT

(76) Inventor: Gregory P. Still, Golden, CO (US)

(21) Appl. No.: 13/179,388

(22) Filed: Jul. 8, 2011

Related U.S. Application Data
(60) Provisional application No. 61/362,550, filed on Jul. 8, 2010.

(51) Int. Cl. A61F 2/42

(52) U.S. Cl. 623/21.18

(57) ABSTRACT

Implants for ankles are disclosed herein. An embodiment of the implant includes a base and a stem. The base includes an upper surface and a lower surface, the upper surface is contoured to contact a distal tibia and the lower surface is contoured to contact a talus. The stem is attachable to the lower surface of the base and protrudes from the base. The stem is implantable into the talus.

This application claims priority to provisional patent application 61/362,550 for PARTIAL ANKLE JOINT REPLACEMENT IMPLANT of Gregory Still, filed on Jul. 8, 2010, which is incorporated herein for all that is disclosed.

### BACKGROUND

Failure of the ankle proximate the tibia and talus can be rather severe. In many situations, the cartilage between the talus and the distal tibia deteriorates, which reduces the motion of the ankle and causes pain in the ankle. Other problems may also cause problems with this joint. In many situations, a total ankle replacement (TAR) is used to alleviate these ankle problems. A TAR requires removal of part of the distal tibia and the part of the talus. Separate implants are then secured to the talus and distal tibia with a bearing or the like located between the implants.

The TARs have several problems and are rather invasive. For example, both the talus and the tibia need to be cut or resurfaced in order to receive their implants. Furthermore, the TAR requires three main components, the implant on the tibia, the implant on the talus, and the bearing located between the two implants. Such a complex device can be complicated to install and may cause two areas, the tibia and talus, that require recovery.

When a problem occurs with a TAR, the TAR may have to be removed. The removal may include removing both implants. Because of the invasive nature of the TAR, there may not be enough bone to attach another implant, therefore, the ankle may have to be fused. In worst case situations, the ankle cannot be repaired and amputation is required.

### BRIEF DESCRIPTION OF THE DRAWINGS

FIG. 1 is a side elevation view of an embodiment of an implant including a base and a separate stem.

FIG. 2 is an anterior view of an ankle with the implant of FIG. 1 located therein.

FIG. 3 is a lateral view of an ankle with the implant of FIG. 1 located there.

FIG. 4 is a medial view of an ankle with the implant of FIG. 1 located therein.

FIG. 5 is a bottom view of the base of the implant of FIG. 1.

FIG. 6 is a top plan view of a filler that may be inserted into the channel of the base of the implants of FIGS. 1 and 5.

FIG. 7 is another embodiment of the bottom of the base of the implant shown in FIG. 1.

FIG. 8 is another embodiment of the base and stem configuration of the implant.

FIG. 9 is a side view of another embodiment of a stem having stabilizing fins.

### DETAILED DESCRIPTION

Embodiments of ankle implants are disclosed herein. More specifically, implants that include a single element located on the talar dome and that contact the distal tibia are disclosed herein. Single element implants are devices that implant solely in the talus and not in the tibia. Accordingly, ankle implants disclosed herein do not require removal of any portions of the distal tibia. In addition, various mechanisms are disclosed that serve to attach the implant to the talus. It is noted that many of the implants disclosed herein may be attached to the dorsal surface of the cancellous bone of the talus.

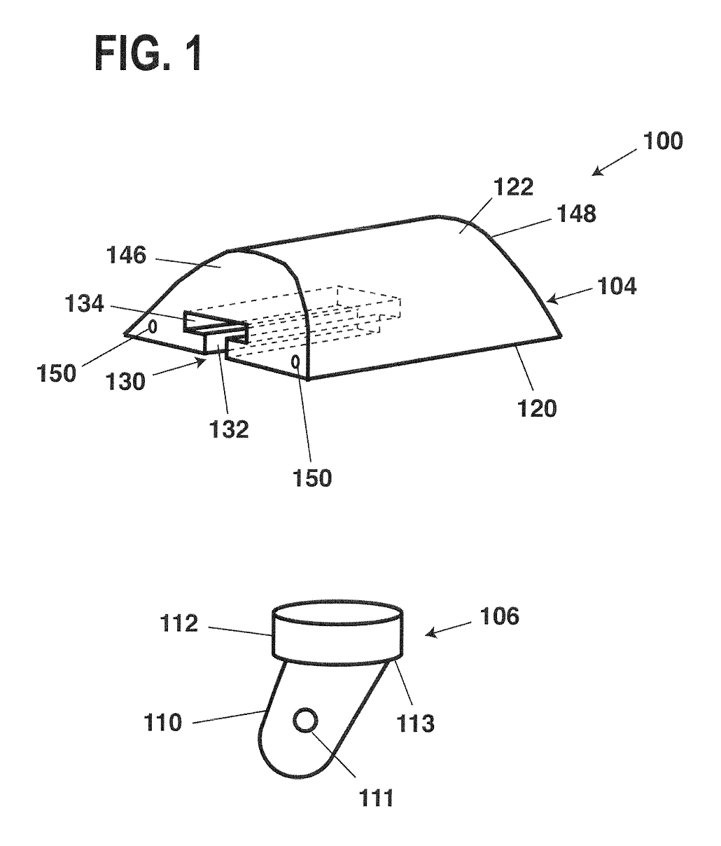

FIG. 1 is a side perspective view of an ankle implant 100. The implant 100 include a base 104 and a stem 106 that is attachable to the base 104. It is noted that the base 104 is shown with sharp edges, however, in some embodiments, the base 104 has substantially rounded edges. As described in greater detail below, the stem 106 may have a plurality of different configurations and is used to secure the base 104 to the talus. The stem 106 has a fin 110 that is inserted into the talus. After insertion into the talus, the talus may grow onto the fin 110 or the fin may be adhered or otherwise attached to the talus. In some embodiments, the fin 110 may have a hole 111 extending through the fin 110. A pin, screw, or other securing device (not shown in FIG. 1) may be placed through the talus and through the hole 111 to secure the stem 106 to the talus.

A mounting portion 112 of the stem 106 is located above the fin 110 and is not located within the talus. The mounting portion 112 has a lower surface 113 that may be located above the talus when the stem 106 is inserted into the talus. The mounting portion 112 is shown as being cylindrical, however, the mounting portion 112 may be in other shapes as described below. The mounting portion is used to affix the base 104 to the stem 106. As described in greater detail below, the fin 110 and the mounting portion 112 may have many different configurations than those shown in FIG. 1.

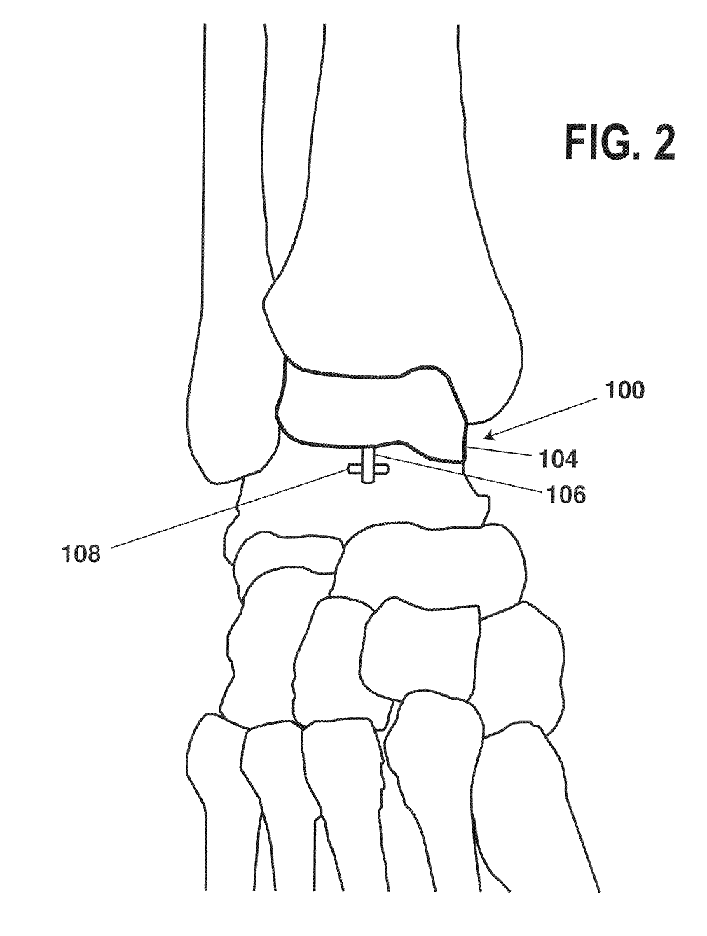

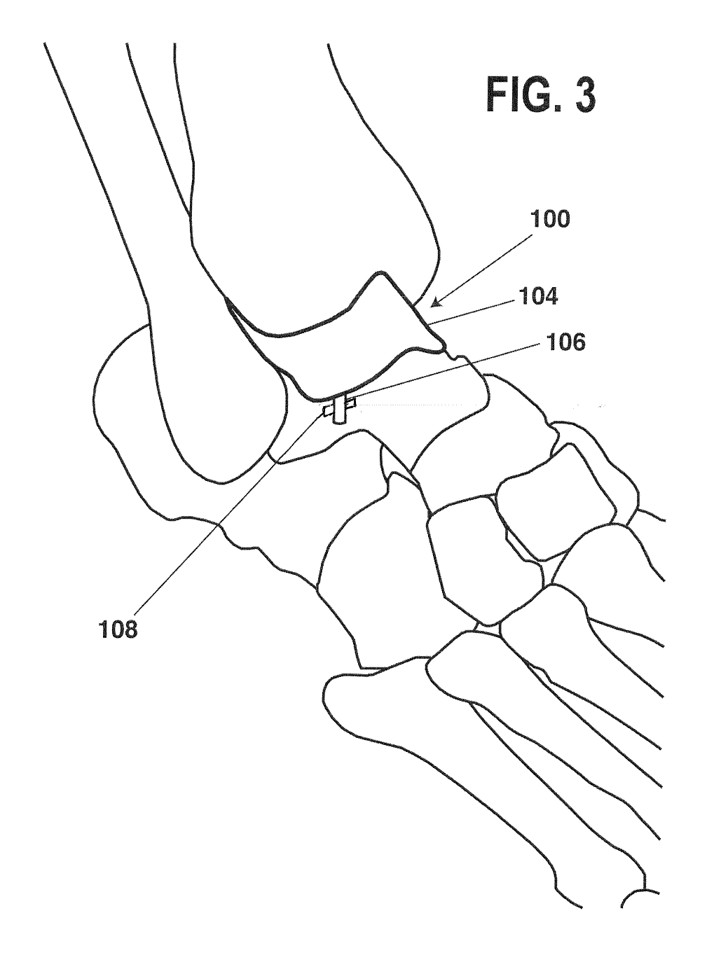

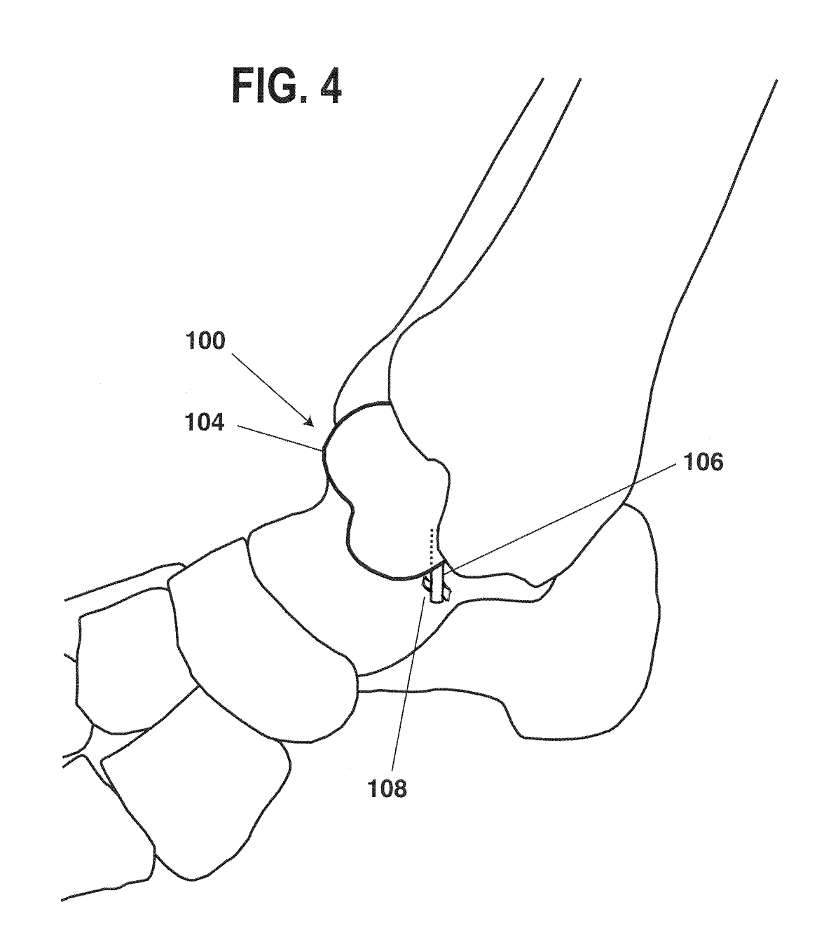

Having described an embodiment of the implant 100, it will now be shown in place in an ankle FIG. 2 is an anterior view of an ankle with the implant 100 located therein. FIG. 3 is a lateral view of an ankle with the implant 100 located therein. FIG. 4 is a medial view of an ankle with the implant 100 located therein. As shown, the implant 100 sets on the talar dome of the talus. The stem 106 is implanted into the talus and, in the embodiments of FIGS. 2-4, is secured via a pin 108 that passes through the talus and the hole 111, FIG. 1. In other embodiments, a screw or other securing device may pass through the hole 111 to secure the stem 106 to the talus.

The base 104 has a lower surface 120 and an upper surface 122. The lower surface 120 is configured to contact the talus or talar dome. In some embodiments, the talus may be cut or otherwise modified to accept the implant 100. More specifically, during installation of the implant 100, the talus may be cut to receive the lower surface 120 of the base 104. In some embodiments, the lower surface 120 may be substantially flat. Accordingly, the talus may be cut or otherwise modified to be substantially flat, which enables the base 104 to fit securely to the talus. In other embodiments, the cartilaginous surface of the talus in the ankle mortise is removed. In these embodiments, the lower surface 120 may be the shape of the surface of the talus with the cartilaginous surface removed.

The lower surface 120 may also include structures or components that are adapted to receive the mounting portion 112 of the stem 106. Accordingly, the mounting portion 112 is able to be affixed to the base 104 by way of the lower surface 120. It is noted that in some embodiments described below, the stem and base are a single unit.

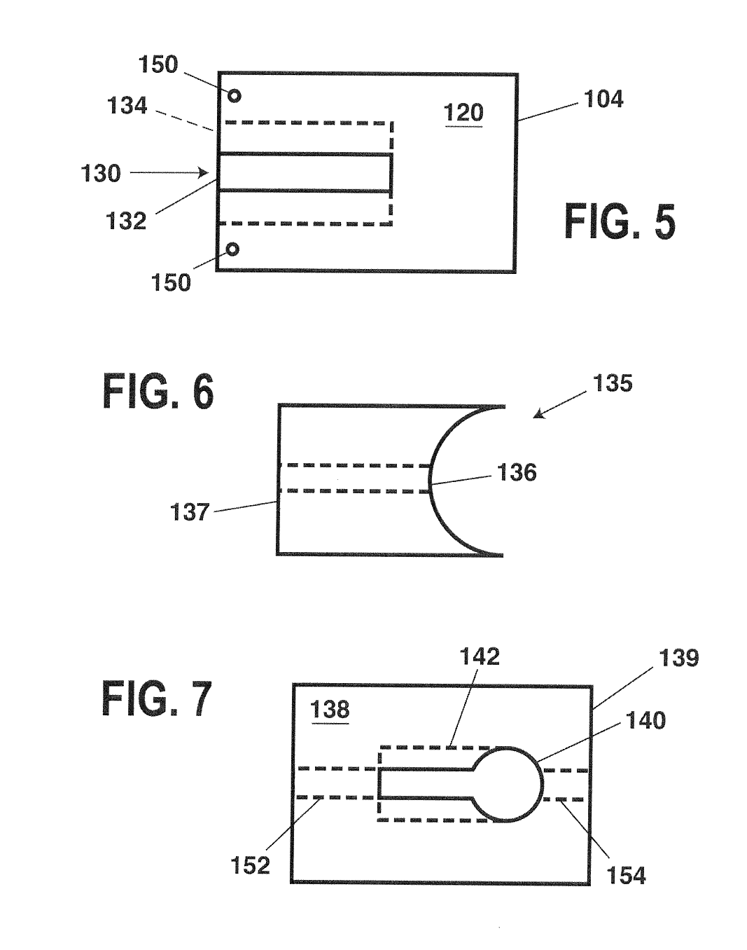

In the embodiment of FIG. 1, the base 104 has a channel 130 formed or cut therein that extends to the lower surface 120. A bottom view of the base 104 showing the channel 130 is also shown in FIG. 5. The channel 130 is sized to receive the mounting portion 112 of the stem 106. The channel has a narrow portion 132 that is adjacent the lower surface 120. The channel also has a wide portion 134 that is adjacent the narrow portion 132 and located opposite the lower surface 120. The mounting portion 112 of the stem 106 is slidable into the wide portion 134 and the fin 110 is slidable into the narrow portion 132. The configuration of the base 104 and the stem 106 enables a surgeon to attach the stem 106 to the talus, then slide the base 104 onto the mounting portion 112 via the channel 130. In some embodiments, the fin 110 is narrow in the proximity of the narrow portion 132 of the channel 130. This fin configuration enables the base to be rotated slightly, which may enhance alignment of the implant 100 to the talus.

FIG. 6 is a top plan view of a filler 135 that may be inserted into the channel 134 after the base 104 is attached to the stem 106. The filler 135 has a first end 136 and an opposite second end 137. The first end 136 is contoured to match the mounting portion 112 of the stem 106. In the embodiment of FIG. 1, the mounting portion 112 is cylindrical, so the contour of the first end 136 is concave with a radius matching the radius of the mounting portion 112. If the mounting portion 112 is square, the first end 136 may be substantially flat. The second end 137 may be contoured to match the exterior surface of the base 104 in the proximity of the channel 130. The contour of the second end 137 removes sharp edges within the empty channel 130. The filler 135 is used to fill the space of the channel 130 that is not occupied by the mounting portion 112. An expansion device (not shown) may be located on the second end 137 to hold the filler 135 in the channel 130. For example, the second end 137 may be split and have a hole to accommodate a screw. When the screw is inserted into the hole, the second end 137 expands, which exerts a force on the channel 130 via the second end 137 to maintain the filler 135 in the channel 130.

The upper surface 122 is shaped to resemble the talar dome. Therefore, the distal tibia may contact the upper surface 122 in the same manner as it contacted the talar dome. It follows that the upper surface 122 is smooth and is made from a material that offers little friction with the tibia, such as titanium or stainless steel. In some embodiments, the upper surface 122 of the base 104 may be the shape of a common talar dome. A surgeon may have several bases 104 that have different shaped upper surfaces that accommodate different shapes and sizes of talar domes. For example, the surgeon may have bases that range in size from 3.25 cm by 3.5 cm to 4.25 cm by 4.5 cm. In other embodiments, images may be made of the talar dome of the recipient of the implant 100. The base 104 may then be molded, machined, or otherwise shaped to match the talar dome of the recipient. Such an embodiment provides for better matching of the distal tibia to the upper surface 122 of the base 104 and may provide for a better fit of the lower surface 122 to the talus.

As set forth above, the implant 100 has a separate stem 106 and base 104. The use of a separate stem 106 enables the stem 106 to be secured to the talus without the base 104. The base 104 may be attached subsequent to the attachment of the stem 106 to the talus. This embodiment of the implant 100 enables the stem 106 to be precisely placed on the talus without interference from the base 104. This embodiment also enables a surgeon to install the implant without separating the talus and tibia as wide as the whole implant 100. Rather, the separation between the talus and tibia only needs to be wide enough to accommodate installation of the stem 106 into the talus. For example, the surgeon may perform a medial malleolar osteotomy to obtain access to the talus. Then a slight separation of the talus and tibia is performed to implant the stem 106 and install the base 104 to the stem 106.

In addition to easy mounting, the implant 100 provides for easy removal should it become necessary. During removal of the implant 100, the base 104 is detached from the stem 106. When the base 104 is detached, the surgeon has greater access to the stem 106, which can then be removed from the talus using conventional techniques.

Having described some embodiments of the implant 100 and methods of inserting the implant, other embodiments of the implant and insertion methods will now be described.

Another embodiment of the lower surface 138 of a base 139 is shown in FIG. 7, which is a bottom view of the base 139. Except for some differences in the lower surface 138, the base 139 is substantially similar to the base 104 of FIG. 1. In the embodiment of FIG. 7, the bottom surface 138 has a hole 140 formed therein. A channel 142 extends from the hole 140. The channel 142 is substantially similar to the channel 130 of FIG. 5. The hole 140 is sized to receive the mounting portion 112 of the stem 106. Once inserted, the mounting portion 112 is able to slide in the channel 142, which secures the base 104 to the stem 106.

In order to secure the base 139 of FIG. 7 to the stem 106, screws or other devices may be located in the base 139 to contact the mounting portion 112. The base 139 may have a first hole 152 and a second hole 154. Both the first hole 152 and the second hole 154 may be threaded to receive screws. After the base 139 is attached to the stem 106, a screw (not shown) may be threaded into the second hole 154. This screw in the second hole 154 keeps the mounting portion 112 fixed relative to the base 104 and prevents movement of the base 104 relative to the stem 106. In some embodiments, a screw (not shown) may be threaded into the first hole 152 to further maintain the position of the base 139 to the stem 106.

Alternative embodiments of the mounting portion 112 may also be used with the implant 100. The mounting portion 112 of the stem 106 has been described herein as being cylindrical. However, the mounting portion 112 may have different shapes. For example, the mounting portion 112 may be a rhombus, or other shape having at least one flat side. The flat side or flat sides of the mounting portion 112 serve to maintain the base 104 in a fixed position relative to the stem 106. More specifically, the flat side or sides may contact the wide portion 134 of the channel 130, FIGS. 1 and 5, which prevents the base 104 from rotating relative to the stem 106. Such a configuration provides for a slightly different installation of the implant 100. For example, the stem 106 is placed in the talus in a manner that the flat portions are oriented in a specific direction because the base 104 will not be able to rotate relative to the stem 106. Accordingly, placement of the stem 106 in the talus is very important. The base 139 of FIG. 7 may be configured to receive the above-described embodiment of the mounting portion 112.

In order to further secure the base 104 to prevent it from moving relative to the talus, screws, pins, or other attachment mechanisms may be used between the base 104 and the talus. The base 104 of FIG. 1 has a first side 146 and a second side 148 that is located opposite the first side 146. The first side 146 and/or the second side 148 may have holes 150 located therein that pass through to the bottom surface 120. Screws or other attaching devices (not shown) may be inserted into the holes 150 and through the base 104 where they are secured into the talus. This securing mechanism lessens the probability that the base 104 will move relative to the talus. Additionally, having the screws or other attachment devices located in the sides 146, 148 of the base 104 reduces the possibility that they will interfere with movement of the tibia on the implant 100.

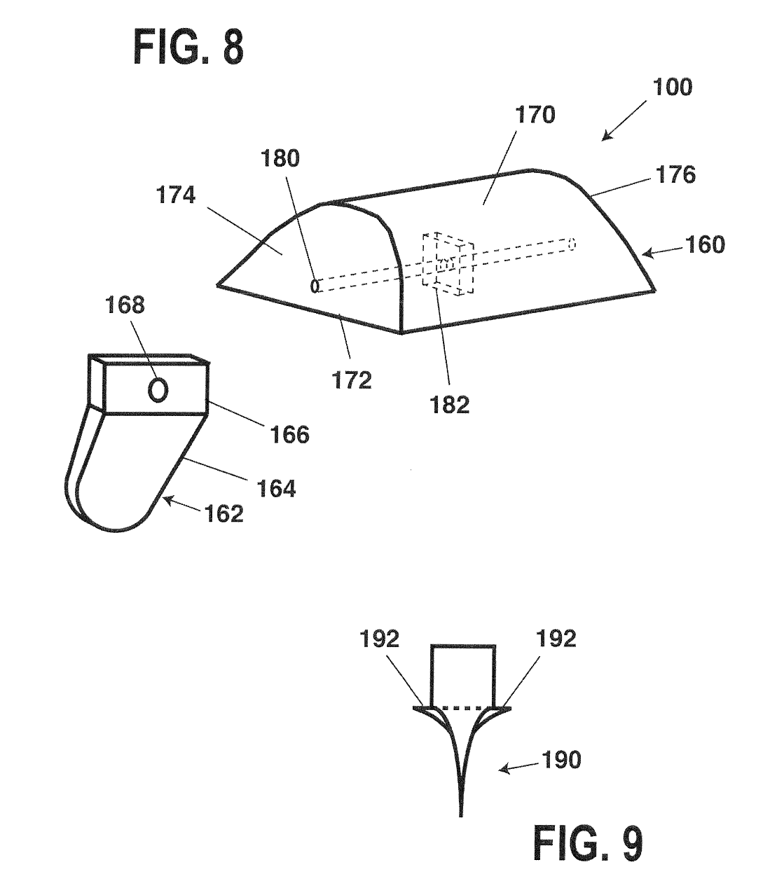

Yet another embodiment of a base 160 and stem 162 is shown in FIG. 8. The stem 162 is similar to the stems described above in that it has a fin 164 and a mounting portion 166. The fin 164 implants into the talus as described above. The mounting portion 166 attaches to the base 160 as described below. The mounting portion 166 has a hole 168 extending through it. As described below, the hole 168 is configured to receive a pin, screw, or the like that secures the stem 162 to the base 160.

The base 160 has an upper surface 170 that is substantially similar to the upper surface 120 of FIG. 1. The base 160 also has a lower surface 172 that may be configured to set on the talus as described above. The base 160 includes a first side 174 and a second side 176, wherein a hole 180 passes between the first side 174 and the second side 176. The lower surface 172 has a cavity 182 formed therein that is sized to receive the mounting portion 166 of the stem 162. The hole 180 passes though the cavity 182. When the mounting portion 166 is inserted into the cavity 182, the hole 168 in the mounting portion aligns with the hole 180 in the base 160. Accordingly, the base 160 may be attached to the stem 162 by inserting a pin or other securing mechanism into the hole 180. For example, part of the hole 180 may be threaded. A pin having threads in the portion aligned with the threads in the hole 180 may be inserted and screwed therein, which secures the stem 162 to the base 160.

FIG. 9 is a side view of another embodiment of a stem 190 having stabilizing fins 192. The stabilizing fins 192 are implanted into the talus and serve to reduce or eliminate rotary motion of the stem 190 in the talus. The embodiment of the stem 190 of FIG. 9 has two stabilizing fins 192 (one is not visible in the figure) that extend substantially perpendicular to the stem 190. Other embodiments of the stem 190 have different numbers of stabilizing fins 192, which is a design choice.

The implants 100 have been described above as having a separate stem and base. Other embodiments are possible wherein the base and stem are a single unit. Single unit implants still have the advantage of having a surface that contacts the tibia. Therefore, the tibia may remain intact. Such an implant follows, for example, the implant 100 of FIG. 1, but has the stem 106 and base 104 as a single unit rather than as individual units.

While illustrative and presently preferred embodiments of the invention have been described in detail herein, it is to be understood that the inventive concepts may be otherwise variously embodied and employed and that the appended claims are intended to be construed to include such variations except insofar as limited by the prior art.

What is claimed is:

1. An implant for an ankle, said implant comprising:
a base, said base comprising an upper surface and a lower surface, said upper surface being contoured to contact a distal tibia, said lower surface being contoured to contact a talus;
a stem, said stem being attachable to said lower surface of said base, and said stem being implantable into a talus.

2. The implant of claim 1, wherein said stem and said base are individual devices.

3. The implant of claim 1, wherein said stem comprises a fin and a mounting portion and wherein said base comprises a channel, said fin being implantable into said talus and said mounting portion being receivable in said channel.

4. The implant of claim 3, wherein said mounting portion is substantially cylindrical.

5. The implant of claim 3, wherein said mounting portion has at least one flat side, said at least one flat side being contactable with at least one side of said channel.

6. The implant of claim 3 and further comprising an securing mechanism that secures said base to said mounting portion.

7. The implant of claim 3, wherein said base has a hole extending from the exterior of said base to said channel, wherein a securing device is insertable into said hole, said securing device contacting said mounting portion when said securing device is in said hole.

8. The implant of claim 7, wherein said securing device is a screw.

9. The implant of claim 3, wherein said bottom surface has a hole located therein, said hole sized to receive said mounting portion and contacting said channel.

10. The implant of claim 1, wherein said talus is modified and wherein said lower surface is contoured to the shape of the modified talus.

11. The implant of claim 1, wherein said lower surface of said base has a cavity formed therein and wherein said stem has a mounting portion, said mounting portion being receivable in said cavity.

12. The implant of claim 11, wherein said base has a hole extending into said cavity and wherein said mounting portion has a hole located therein and wherein when said mounting portion is located in said cavity, said hole in said base is aligned with said hole in said mounting portion.

13. The implant of claim 12, wherein a pin is receivable in said hole in said base and said hole in said mounting portion.

14. The implant of claim 12, wherein a screw is receivable in said hole in said base and said hole in said mounting portion.

15. A method if inserting an ankle implant, said method comprising:
exposing the talar dome;
inserting a stem into the talar dome;
attaching a base to said stem, said base having an upper surface that contacts the tibia and a lower surface that contacts the talar dome.

16. The method of claim 15, wherein said base has a cavity in said lower surface and wherein said stem has a mounting portion that is receivable in said cavity, wherein said method further comprises locating said mounting portion in said cavity.

17. The method of claim 16, wherein said base has a hole extending to said cavity and wherein said method further comprises placing a securing device in said hole, wherein said securing device contacts said mounting portion.

18. The method of claim 17, wherein said mounting portion has a hole that is aligned with said hole in said base when said mounting portion is located in said cavity, and wherein said method further comprises placing a securing device in said hole in said base and into said hole in said mounting portion.

19. The method of claim 16, wherein said cavity is a channel and wherein said mounting portion is slidable in said cavity.

20. The method of claim 15, wherein said stem comprises a fin having a hole located therein and wherein said method further comprises inserting said fin into said talus and placing a securing device in said talus and through said hole in said fin.
